(19) United States
(12) Patent Application Publication
(10) Pub. No.: US 2016/0175153 A1
(43) Pub. Date: Jun. 23, 2016

(54) KERATOCONUS TREATMENT

(71) Applicant: Joerg H. KRUMEICH, Bochum (DE)

(72) Inventor: Joerg H. KRUMEICH, Bochum (DE)

(21) Appl. No.: 14/579,022

(22) Filed: Dec. 22, 2014

(51) Int. Cl. A61F 9/008

(52) U.S. Cl. CPC A61F 9/00825; A61F 9/008; A61F 2009/00872; A61F 2009/00893

(57) ABSTRACT

The invention concerns a method for treating keratoconus, wherein a laser beam is directed to the cornea (1) of the eye of a patient suffering from keratoconus and wherein a circular laser-cut (14) is made within the stroma layer (7) of cornea (1) around the optical axis (13) of the eye creating an annular scar within the stroma layer (7) to stabilize the diseased cornea (1). The method is an improvement of the so-called circular keratotomy (CKT) performed with mechanical incision. It does not open the surface of the eye so that no infection may occur. No suture is needed that has to be watched, possibly renewed and removed under the mircoscope. It allows a fast treatment and safe postoperative recovery and typically needs no anesthesia.

The invention concerns a method for treating keratoconus.

Keratoconus is a degenerative eye disease. The cornea assumes a conical shape that is in contrast to the natural curvature of the cornea. This leads to substantial distortion of vision, e.g. multiple images and streaking. Since it is a rare eye disease, which becomes manifest at adolescent age, a keratoconus quite often is not diagnosed correctly or at a late stage. The disease deformes the cornea which then assumes a conical shape and becomes thinner than normal. Progress of the disease is typically classified into four stages according to the table of Krumeich/Amsler that need different kinds of treatment.

The cornea consists of several layers wherein the stroma consisting of collagen fibrils is the thick transparent middle layer. Up to 90% of the corneal thickness is composed of stroma.

The exterior layer of the cornea is formed by the cornea epithelium, a thin multicellular epithelium layer. The layer between the epithelium layer and and the corneal stroma is called Bowman's membrane.

To the posterior side the layer abutting the stroma is called Descemet's membrane followed by the corneal endothelium.

In most cases the symptoms of keratoconus are simply treated by glasses or contact lenses at least in early stages. In particular rigid gas-permeable (RGP) lenses may be used that act by covering over the cone of the eye, thereby creating the effect of an evenly dome-shaped convex lens. Sometimes also scleral lenses are used that cover a greater portion of the eye's surface. Although patients suffering from keratoconus may be satisfied with the vision achieved with contact lenses it has to be borne in mind that with this prescription the disease is not actually treated but symptoms of the disease are merely attenuated. This may have the effect that the disease itself progresses to higher stages making surgical options for correction even more difficult.

One possibility for the treatment of keratoconus is corneal collagen cross-linking (CXL). Riboflavin is applied to the eye and activated by UV-A light. The riboflavin causes new bonds to be formed across adjacent collagen strands in the stroma layer of the cornea. The corneal mechanical strength may thereby be enhanced because the cross-linked collagen is less deformable. However, long-time studies for CXL treatment are missing. Some practitioners report about indications that cornea transplantation (keratoplasty) may no longer be possible after a CXL treatment (Krumeich et al., Cornea 2014, 33, 313-316).

In epikeratophakia (EPI) the corneal epithelium is removed and a disk of a donor cornea is grafted on top of the cornea of the patient. The disk is refractively adapted to the patient's refraction, typically with the help of an excimer laser.

It becomes necessary to treat a keratoconus in its advanced stages by a corneal transplantation, when vision correction is no longer possible and the cornea has become very thin. This procedure also called keratoplasty uses a donor cornea to replace the patient's cornea completely (penetrating keratoplasty). Alternatively only the outer layers of the cornea are replaced (lamellar keratoplasty) and the patient's Descemet's membrane and Endothelium are preserved.

Another procedure for treatment of initial keratoconus is known as circular keratotomy (CKT). Typically an incision of a diameter of about 7 mm is made into the cornea around the optical axis to cause a circular scar. The cornea is fixed by a suction ring while the surface of the cornea is pressed against a spherical glass body inside the trephine and thereby rounded. The depth of the incision is around 90% of the total cornea thickness. The incision is sewed by a 10x0 nylon suture. The procedure can be applied in keratoconus patients if the corneal thickness is not too low.

By the circular keratotomy procedure a circular scar is generated leading to a strengthening of the cornea. CKT is performed by a mechanical trepan system. According to a statistical evaluation the results are very good; from 246 treated eyes 83% resulted in total stability of the cornea. 73% showed improvement regarding visual acuity. However, making the incision with a trephining system, i.e. a round hollow blade, disadvantages must be accepted. The incision must be sutured very regularly using high-tech measurements of the surface radii during the suture. Further the incision may be prone to an infection, the stitches may pull through due to the weak and thinned-out tissue. Further the sutures have to be replaced when pulling through which indeed may be cumberson for the patient.

In view of the fact that keratoconus is accompanied by progressive thinning of the cornea, typical laser procedures for correction of ametropia like PRK (photorefractive keratectomy) or LASIK (laser assisted in-situ keratomileusis) are contraindicated because they lead to additional structural weakening of the cornea. Surprisingly, according to the invention it turned out to be possible to treat keratoconus by circular keratotomy with the help of a laser.

According to the invention a method for treating keratoconus is provided, wherein a laser beam is directed to the cornea of the eye of a patient suffering from keratoconus. A circular laser-cut of 360° is made within the stroma layer of the cornea while Bowman's as well as Descemet's membrane are left intact. The circular laser-cut is made around the optical axis of the eye, i.e. the center of the circular laser-cut corresponds to the center of the eye or the center of the pupil. The healing of this laser-cut results in an annular scar within the stroma layer surrounding the optical axis of the eye and stabilizing the entire cornea.

In comparison to mechanical CKT the procedure is totally painless, hence, no anesthesia is needed. Healing takes place more quickly, the patient may be able to go back to work within a matter of 1 to 2 days. The risk of infection is very low, because all outer structures of the eye remain intact.

The procedure differs from typical laser therapies for correction of refractive errors. PRK and LASIK are used to modify the shape of the cornea in order to thereby change its power and compensate the ametropia. Tissue of the cornea is removed by ablation. In a PRK procedure ablation commences on the surface layers of the cornea, i.e. epithelium and Bowman's membrane while in a LASIK procedure an incision is made underneath Bowman's membrane whereupon this layer can be folded aside to form a so-called “flap”. Afterwards the stroma is ablated. At the end of the LASIK procedure the flap is folded back to the cornea. However, neither PRK nor LASIK can be used for the treatment of keratoconus as weakening of the tissue would ensure. Furthermore, neither PRK nor LASIK have a circular incision of 360°.

Although a laser treatment method for keratoconus as well as several other eye dysfunctions is described in WO 2014/087250 A2, however, this document follows the concept of femto multi shooting on the cornea or on the lens by distributing multiple laser pulses separated from each other, without creating a cut. Instead, the corneal stroma or the lens fibers are structurally modified by creating micro vacuoles or micro cavities on the tissues of the cornea or the lens. This method decreases links between collagen fibers of the stroma and is in total contrast to attempts like CXL to strengthen the connectivity between collagen fibers.

It was quite surprising that a CKT procedure may be performed with the help of a laser since those skilled in the art know that a contraindication applies for keratoconus. The reason is that the cornea of a keratoconus patient is thinned out and a laser treatment may even worsen this condition. However, the present invention stabilizes the surface radii, improves visual acuity, decreases irregular configuration by generating a scar by a circular cut symmetrically around the optical axis. It is possible to limit the cut just to the stroma of the cornea by focusing the laser beam. The focal spot may be specified by the surgeon and is located within the stroma layer, in contrast to mechanical incisions which affect the outer cornea layers also. As far as the application mentions laser beams it should be obvious for those skilled in the art that it is possible to apply a multitude of beams or laser pulses.

According to a preferred embodiment of the invention the cut is made inside the stroma leaving about 70% of the corneal tissue unaltered. The cut extends from below Bowman's to close to Descemet's membrane. The cut reaches 50 to 95% of the stroma layer thickness. Even more preferred is a cut extending from 70 to 95 or 80 to 95% of the stroma layer thickness. Most preferred is the cuts extrension of 90% of the stroma layer thickness.

Preferably, the laser used according to the invention works with ultrashort pulses. In particular, the laser is a femtosecond laser as used e.g. for the flap creation in LASIK technology or corneal transplants. Femtosecond lasers are available from different companies like Intralase Corp./Advanced Medical Optics, USA; Carl Zeiss Meditec, Germany; Ziemer Ophthalmic Systems AG, Switzerland, or Bausch+Lomb, USA.

For the diameter of the circular cut within into the stroma layer of the cornea a range between 4 and 10 mm is preferred. Even more preferred are ranges between 5 and 9 mm or between 6 and 8 mm. Most preferred is a diameter of 7 mm.

The present invention will now be explained in greater detail, using a principal example. For this purpose the enclosed drawing shows:

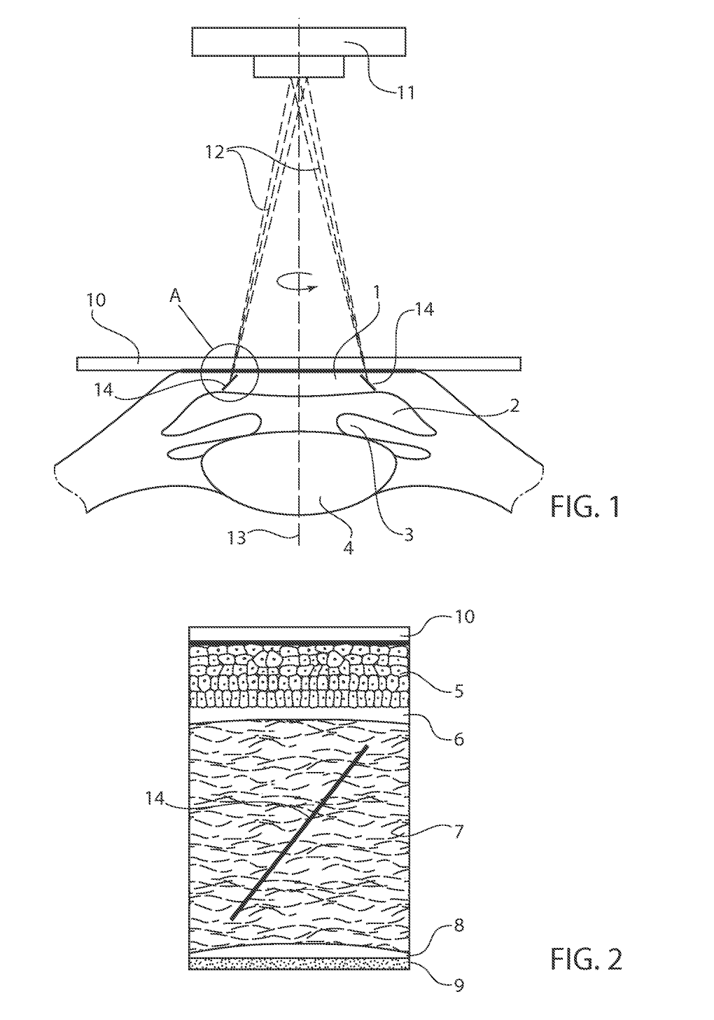

FIG. 1: a cornea in cross-section during laser treatment;

FIG. 2: enlarged the detail A of FIG. 1.

In FIG. 1, the cornea of the eye is designated with the reference number 1. Under the cornea 1 are situated
the anterior chamber 2
the iris 3
and the lens 4
   of the eye.

As can be taken from FIG. 2, the cornea 1 has several layers, especially
the epithelium layer 5
the Bowman's layer 6
the stoma layer 7
the Descemet's layer 8
and the endothelium layer 9

The stroma layer 7 has up to 90% of the thickness of the entire thickness of the cornea 1.

For the treatment according to the invention, the surface of the cornea 1 is flattened by means of a flat glass plate 10, pressed against the cornea 1 as illustrated in FIG. 1. Above the glass plate, a laser source 11 (preferably an femtosecond laser) is arranged, which generates a laser beam 12 directed to the cornea 1.

This laser beam 12 is focused to the stroma layer 7 and guided along a 360° circular path around the optical axis 13 of the eye, so as to produce an annular laser-cut 14 within the stroma layer 7 leaving the epithelium layer 5, the Bowman's layer 6, the Deccemet's layer 8 and the endothelium layer 9 intact.

After healing the annular laser-cut 14 results in an annular scar within the stroma layer 7 so as to stabilize the diseased cornea 1.

1. A method for treating keratoconus, wherein a laser beam is directed to the cornea (1) of the eye and wherein a circular laser-cut (14) is made within the stroma layer (7), of the cornea (1) around the optical axis (13) of the eye creating an annular scar within the stroma layer (7) to stabilize the diseased cornea (1).

2. The method according to claim 1, wherein the laser-cut (14) is made in an area of 50 to 95% of the stroma layer thickness.

3. The method according to claim 2, wherein the laser-cut (14) is made in an area of 70 to 95% of the stroma layer thickness.

4. The method according to claim 3, wherein the laser-cut (14) is made in an area of 80 to 95% of the stroma layer thickness.

5. The method according to claim 4, wherein the laser-cut (14) is made in an area of 90% of the corneal thickness.

6. The method according to claim 1, wherein a femtosecond laser is used.

7. The method according to claim 1, wherein the circular laser-cut (14) within the stroma layer (7) of the cornea (1) has a diameter of 4 to 10 mm.

8. The method according to claim 7, wherein the circular laser-cut (14) within the stroma layer (7) of the cornea (1) has a diameter of 5 to 9 mm.

9. The method according to claim 8, wherein the circular laser-cut (14) within the stroma layer (7) of the cornea (1) has a diameter of 6 to 8 mm.

10. The method according to claim 9, wherein the circular laser-cut (14) within the stroma layer (17) of the cornea (1) has a diameter of 7 mm.
